(19) United States
(12) Patent Application Publication
(10) Pub. No.: US 2024/0292880 A1
(43) Pub. Date: Sep. 5, 2024

(54) WATER-BASED ATOMIZING AGENT, WATER-BASED ATOMIZING SUBSTANCE FOR SPECIFIED PURPOSE, CARTOMIZER AND ATOMIZING DEVICE

(71) Applicants: SHENZHEN FUMIAO BIOTECHNOLOGY CO., LTD, Shenzhen (CN); AQUIOS LABS LTD.

(72) Inventor: Yecheng KUANG, Shenzhen (CN)

(73) Assignees: SHENZHEN FUMIAO BIOTECHNOLOGY CO., LTD., Shenzhen (CN); AQUIOS LABS LTD., (omitted)

(21) Appl. No.: 18/661,572

(22) Filed: May 11, 2024

Related U.S. Application Data
(63) Continuation of application No. PCT/CN2022/116832, filed on Sep. 2, 2022.

(30) Foreign Application Priority Data
Dec. 22, 2021 (CN) 202111583785.6

(51) Int. Cl. A24B 15/167; A24B 15/32; A24F 40/10; A61M 15/06

(52) U.S. Cl. CPC A24B 15/167; A24B 15/32; A24F 40/10; A61M 15/06

(57) ABSTRACT

Disclosed are a water-based atomizing agent, a water-based atomizing substance for a specified purpose, a cartomizer, and an atomizing device. The water-based atomizing agent comprises a mixed matrix and an active agent for improving a surface activity of the mixed matrix.

### CROSS-REFERENCE TO RELATED APPLICATION

This application is a continuation of International Application No. PCT/CN2022/116832, filed on Sep. 2, 2022, which claims priority to Chinese Patent Application No. 202111583785.6, filed on Dec. 22, 2021. The entire contents of each of the above-referenced applications are expressly incorporated herein by reference.

### TECHNICAL FIELD

The present disclosure relates to the field of atomization, and particularly to a water-based atomizing agent, a water-based atomizing substance for a specified purpose, a cartomizer, and an atomizing device.

### BACKGROUND

With the rapid development of the field of electronic atomization in recent years and the gradually expanded cognition of the electronic atomizing device of people, the market supervision and regulations in the field of electronic atomization under the national legal system are becoming stricter. Under this background, it is urgent to further innovate and develop an atomizing liquid that is less harmful to the human body, can bring better taste experience, and complies with laws and regulations and supervision requirements.

There is usually no water in a traditional atomizing liquid, which is mainly because the atomizing liquid containing water is difficult to produce sufficient smoke for a user to inhale, and the water is easy to leak out of an atomizer storing the atomizing liquid due to a low viscosity, resulting in liquid leakage. However, the atomizing liquid rich in water has a lower viscosity than the traditional atomizing liquid, and a required atomization temperature can be lower, so that the user may have a more comfortable and refreshing taste, and there are less harmful substances produced during atomization.

It is known that an atomizing liquid containing water and sugar alcohol has been developed, which can produce smoke for human to inhale after being heated at a certain temperature. However, further research has found that the atomizing liquid containing water and sugar alcohol has a poor fluidity after being mixed and heated, E-liquid shortage is easy to occur around a heating assembly of the atomizer, scorching is easy to occur during heating, sometimes failing to produce sufficient smoke and affecting the taste experience of the user due to the scorching.

Under such a demand background, it is necessary to develop an atomizing liquid which can provide a refreshing taste and have a low viscosity, and can produce sufficient smoke during atomization and further reduce the harmful substances produced during atomization.

### SUMMARY

For this purpose, the present disclosure aims to solve at least one of the above technical problems.

According to some embodiments, the present disclosure provides a water-based atomizing agent, comprising:
a mixed matrix, and
an active agent, wherein the active agent is used for improving a surface activity of the mixed matrix.

According to the present disclosure, the active agent is added in the water-based atomizing agent, which can reduce the surface activity of the water-based atomizing agent, thus improving the atomization efficiency and reducing the atomization temperature.

According to an embodiment of the present disclosure, the mixed matrix in the water-based atomizing agent comprises a water matrix and an alcohol matrix. The water matrix is used for producing smoke and dissolving an essence and other substances, and the alcohol matrix is used for dissolving an essence and preventing corrosion and coagulation.

According to an embodiment of the present disclosure, the alcohol matrix is polyol; and the polyol comprises ethylene glycol, propylene glycol, glycerol, pentaerythritol, or a combination thereof.

According to an embodiment of the present disclosure, the alcohol matrix is propylene glycol, glycerol, or a combination thereof.

According to the present disclosure, the mixed matrix of the water-based atomizing agent comprises the alcohol matrix, and the alcohol matrix is preferably the polyol, which can improve the fluidity of the atomizing agent, thus improving atomization and taste (of an electronic cigarette).

According to an embodiment of the present disclosure, the water matrix comprises water and sugar alcohol.

According to an embodiment of the present disclosure, the sugar alcohol comprises erythritol, xylitol, mannitol, sorbitol, or a combination thereof, preferably, the sugar alcohol comprises erythritol, xylitol, mannitol, sorbitol, or a combination thereof, and most preferably, the sugar alcohol is xylitol.

According to the present disclosure, the water matrix of the water-based atomizing agent comprises sugar alcohol, which can improve the viscosity of the atomizing agent, thus increasing the smoke volume and reducing liquid leakage.

According to an embodiment of the present disclosure, wherein:
a mass percentage of the active agent in the water-based atomizing agent is 0.01% to 20%; preferably, the mass percentage of the active agent in the water-based atomizing agent is 0.05% to 15%;
preferably, the mass percentage of the active agent in the water-based atomizing agent is 0.1% to 10%;
preferably, the mass percentage of the active agent in the water-based atomizing agent is 0.2% to 5%;
preferably, the mass percentage of the active agent in the water-based atomizing agent is 0.5% to 3%; and
preferably, the mass percentage of the active agent in the water-based atomizing agent is 1% to 2%.

According to an embodiment of the present disclosure, the water-based atomizing agent consists of the mixed matrix and the active agent.

According to the water-based atomizing agent of the present disclosure, the mixed matrix comprises:
10 to 94 parts by weight of water matrix; and 6 to 90 parts by weight of alcohol matrix;
preferably, 20 to 80 parts by weight of water matrix; and 20 to 80 parts by weight of alcohol matrix;
preferably, 30 to 80 parts by weight of water matrix; and 20 to 70 parts by weight of alcohol matrix;
preferably, 40 to 80 parts by weight of water matrix; and 20 to 60 parts by weight of alcohol matrix;
preferably, 50 to 80 parts by weight of water matrix; and 20 to 50 parts by weight of alcohol matrix;
preferably, 60 to 80 parts by weight of water matrix; and 20 to 40 parts by weight of alcohol matrix; and
preferably, 70 parts by weight of water matrix; and 30 parts by weight of alcohol matrix.

According to an embodiment of the present disclosure, the mixed matrix includes the water matrix and the alcohol matrix.

According to an embodiment of the present disclosure, the water matrix comprises: 60% to 99% by mass percentage of water and 1% to 40% by mass percentage of sugar alcohol;
preferably, 60% to 90% by mass percentage of water and 10% to 40% by mass percentage of sugar alcohol;
preferably, 60% to 80% by mass percentage of water and 20% to 40% by mass percentage of sugar alcohol;
preferably, 60% to 70% by mass percentage of water and 30% to 40% by mass percentage of sugar alcohol; and
preferably, 67% by mass percentage of water and 33% by mass percentage of sugar alcohol.

According to an embodiment of the present disclosure, a surface tension of the active agent is smaller than that of the mixed matrix.

In some embodiments, the active agent is at least one selected from the group consisting of phenethyl alcohol, ethyl lactate, ethyl acetate, and ethanol; and more preferably, the active agent is ethanol.

According to an embodiment of the present disclosure, the water-based atomizing agent is in a liquid state, a semi-solid state, or a solid state.

In a second aspect, the present disclosure provides a water-based atomizing substance for a specified purpose, comprising:
the water-based atomizing agent according to the present disclosure as described above; and
an additive, wherein the additive is used for personal consumption or a medical purpose.

According to the present disclosure, there is no special limitation on the additive, and the substances capable of being matched with the water-based atomizing agent to use as water-based atomizing substances all belong to the scope of the present disclosure.

According to an embodiment of the present disclosure, the personal consumption comprises an electronic cigarette.

According to an embodiment of the present disclosure, the medical purpose refers to an active ingredient suitable for medical use and/or a composition containing related auxiliaries.

According to an embodiment of the present disclosure, the additive contains a natural and/or synthetic specific ingredient.

Preferably, the specific ingredient is an herbal extract.

More preferably, the specific ingredient is nicotine or a derivative thereof.

According to an embodiment of the present disclosure, the water-based atomizing agent further comprises a flavoring agent.

In a third aspect, the present disclosure provides a cartomizer comprising at least one cartomizer chamber, wherein the cartomizer chamber is filled with the water-based atomizing agent according to the present disclosure as described above, or the water-based atomizing substance for a specific purpose according to the present disclosure above.

In a fourth aspect, the present disclosure provides an atomizing device comprising the cartomizer according to the present disclosure above; preferably, the atomizing device is a medical instrument or a personal consumption appliance; and more preferably, the personal consumption appliance is an electronic cigarette.

In a fifth aspect, the present disclosure provides use of the water-based atomizing agent or the water-based atomizing substance for a specific purpose according to the present disclosure as described above in a personal consumption appliance or a medical field; and preferably, the personal consumption appliance is an electronic cigarette.

In a sixth aspect, the present disclosure provides a preparation method of the water-based atomizing agent according to the present disclosure above, comprising:
preparing the mixed matrix; and adding the active agent.

Preferably, the method comprises:
dissolving the sugar alcohol in water to obtain the water matrix;
mixing the polyol with the water matrix to obtain the mixed matrix; and
mixing the mixed matrix with the active agent to obtain the water-based atomizing agent.

According to the present disclosure, the temperature of the preparation method is not particularly limited, and the temperature is preferably 5° C. to 45° C., more preferably 10° C. to 40° C., and most preferably, the temperature is room temperature, i.e., 20° C. to 30° C.

According to the present disclosure, the mixing method in the preparation method is not particularly limited, and a specific mixing method comprises magnetic stirring for mixing, mechanical stirring for mixing, ultrasonic mixing, or a combination thereof, and preferably the mixing is well-mixed.

In a seventh aspect, the present disclosure provides an atomization method, comprising using the water-based atomizing agent or the water-based atomizing substance for a specific purpose or the cartomizer or the atomizing device according to the present disclosure above.

According to an embodiment of the present disclosure, the method comprises heating and/or ultrasonic processing.

According to an embodiment of the present disclosure, the heating is one or more selected from the group consisting of conduction heating, radiation heating, and induction heating.

It can be seen from the technical solutions above that the present disclosure has the following advantages:
the present disclosure provides a water-based atomizing agent, since the water-based atomizing agent comprises a mixed matrix capable of producing smoke and an active agent which is capable of improving the surface activity of the mixed matrix, reducing the surface tension and the atomization temperature, reducing the production of harmful substances in the smoke and improving the fluidity of the mixed matrix, sufficient atomizing liquid can be continuously supplied around the atomizing assembly of the atomizer. The water-based atomizing agent can produce sufficient smoke under environmental conditions such as heating or vibration for a user to inhale, and due to a high water content in the water-based atomizing agent, the taste of the smoke is refreshing, thus improving the taste of the smoke or a therapeutic effect of the water-based atomizing substance on the human after the water-based atomizing substance enters the human body.

### BRIEF DESCRIPTION OF DRAWINGS

FIG. 1 is a diagram showing a relationship between the surface tension of the water-based atomizing agent and the proportion of the active agent in the water-based atomizing agent according to the embodiments of the present disclosure; and

FIG. 2 is a diagram showing a relationship between the total particulate matters and the proportion of sugar alcohol in the water matrix according to the embodiments of the present disclosure.

### DETAILED DESCRIPTION

The present disclosure provides a water-based atomizing agent, a water-based atomizing substance for a specified purpose, a cartomizer, and an atomizing device, which can produce smoke with refreshing taste, sufficient smoke volume, and less harm to human, thus reducing the production of harmful substances in the smoke and improving the taste experience of the smoker.

In an embodiment of the present disclosure, the ingredients of the water-based atomizing agent comprise a mixed matrix and an active agent used for improving a surface activity of the mixed matrix. The mixed matrix is capable of producing smoke under certain conditions. For example, the mixed matrix may be heated and produce smoke under an environmental condition that the water-based atomizing agent is heated to a certain temperature, or the mixed matrix may produce smoke when the water-based atomizing agent is subjected to high-frequency vibration such as ultrasonic waves. For certain conditions for producing the smoke, they are not limited herein.

Since the water-based atomizing agent comprises the mixed matrix capable of producing smoke and the active agent capable of improving the surface activity of the mixed matrix and improving the fluidity of the mixed matrix, sufficient atomizing liquid can be continuously supplied around the atomizing assembly of the atomizer. The water-based atomizing agent can produce sufficient smoke under conditions such as heating or vibration for a user to inhale, and due to a high water content in the water-based atomizing agent, the taste of the smoke is refreshing, thus improving the taste of the smoke or the therapeutic effect of the water-based atomizing agent on a human after the water-based atomizing agent enters the human body.

In the embodiment of the present disclosure, a mass percentage of the active agent in the water-based atomizing agent may be 0.01% to 20%.

It should be noted that, the active agent may be at least one selected from the group consisting of phenethyl alcohol, ethyl lactate, ethyl acetate, and ethanol, which is not specifically limited herein. Preferably, the active agent may be ethanol.

Preferably, the mass percentage of the active agent in the water-based atomizing agent may be 0.05% to 15%.

Preferably, the mass percentage of the active agent in the water-based atomizing agent may be 0.1% to 10%.

Preferably, the mass percentage of the active agent in the water-based atomizing agent may be 0.2% to 5%.

Preferably, the mass percentage of the active agent in the water-based atomizing agent may be 0.5% to 3%.

Preferably, the mass percentage of the active agent in the water-based atomizing agent may be 1% to 2%.

In the embodiment of the present disclosure, the mixed matrix may comprise:
10 to 94 parts by weight of water matrix; and 6 to 90 parts by weight of alcohol matrix;
preferably, 20 to 80 parts by weight of water matrix; and 20 to 80 parts by weight of alcohol matrix;
preferably, 30 to 80 parts by weight of water matrix; and 20 to 70 parts by weight of alcohol matrix;
preferably, 40 to 80 parts by weight of water matrix; and 20 to 60 parts by weight of alcohol matrix;
preferably, 50 to 80 parts by weight of water matrix; and 20 to 50 parts by weight of alcohol matrix;
preferably, 60 to 80 parts by weight of water matrix; and 20 to 40 parts by weight of alcohol matrix; and
more preferably, 70 parts by weight of water matrix; and 30 parts by weight of alcohol matrix.

It should be noted that, the water matrix is used for producing smoke and dissolving an essence and other substances, and the alcohol matrix is used for dissolving an essence, preventing corrosion, and inhibiting coagulation.

It should be noted that, the alcohol matrix may comprise at least one of propylene glycol and glycerol, which is not specifically limited herein. In the embodiment of the present disclosure, the water matrix may comprise:
60% to 99% by mass percentage of water and 1% to 40% by mass percentage of sugar alcohol;
preferably, 60% to 90% by mass percentage of water and 10% to 40% by mass percentage of sugar alcohol;
preferably, 60% to 80% by mass percentage of water and 20% to 40% by mass percentage of sugar alcohol;
preferably, 60% to 70% by mass percentage of water and 30% to 40% by mass percentage of sugar alcohol; and
more preferably, 67% by mass percentage of water and 33% by mass percentage of sugar alcohol.

It should be noted that, sugar alcohol may be at least one selected form the group consisting of erythritol, xylitol, mannitol, and sorbitol; and preferably, sugar alcohol is xylitol.

Further, the surface tension of the active agent is smaller than that of water. In the embodiment of the present disclosure, if there is no active agent in the water-based atomizing agent, when the water content of the water-based atomizing agent is high, the mixed matrix used for producing smoke is wrapped by water therein due to the large surface tension of the water, so that the water-based atomizing agent is difficult to quickly flow to the heating assembly or the ultrasonic assembly of the atomizer, resulting in incapability of continuously obtaining sufficient water-based atomizing agent to produce smoke when the heating assembly heats and atomizes the water-based atomizing agent or the ultrasonic assembly vibrates and atomizes the water-based atomizing agent, which easily leads to E-liquid shortage of the heating assembly or the ultrasonic assembly during atomization of the water-based atomizing agent. Therefore, scorching occurs during atomization, which not only affects the taste of the smoke or the therapeutic effect of the smoke, but also easily damages atomization equipment and shortens a service life of the atomization equipment. Therefore, in the embodiment of the present disclosure, since the active agent such as ethanol is added in the water-based atomizing agent, and the surface tension of the active agent such as ethanol is far smaller than that of the water, the overall surface tension of the water-based atomizing agent is reduced. The mixed matrix can flow more easily and quickly to the periphery of the heating assembly or the ultrasonic assembly due to the reduced surface tension, so that E-liquid shortage is not easy to occur around the heating assembly or the ultrasonic assembly. In this way, when the water-based atomizing agent produces smoke, the scorching is greatly reduced or even eliminated during atomization, thus improving the taste of the smoke and the therapeutic effect of the smoke, ensuring that the atomization equipment is not easily damaged, and prolonging a service life of the atomization equipment for atomizing the water-based atomizing agent.

Further, the surface tension of the active agent is smaller than that of the mixed matrix.

It should be noted that, the mixed matrix may be a fully mixed matrix of water and xylitol, or a fully mixed matrix of water and other sugar alcohols. However, in the embodiment of the present disclosure, the fully mixed matrix of water and xylitol can produce a lot of smoke during atomization, and the experimental result shows that the smoke volume produced by the fully mixed matrix of water and xylitol is obviously more than that of the fully mixed matrix of water and other sugar alcohols.

In addition, the water-based atomizing agent in the embodiment of the present disclosure may be in a liquid state, a semi-solid state, or a solid state, which is not specifically limited herein.

In the embodiment of the present disclosure, the surface tension of the active agent such as the ethanol is smaller than that of the mixed matrix of water and xylitol mixed fully, which can ensure that the water-based atomizing agent fully, quickly, and smoothly flows to the periphery of the heating assembly or the ultrasonic assembly, thus being fully atomized during atomization.

It should be noted that, the water-based atomizing agent in the embodiment of the present disclosure may be E-liquid used in the atomizing device. Moreover, the E-liquid rich in water is usually called water-based E-liquid in the atomizing device industry. The water-based atomizing agent in the embodiment of the present disclosure may also be a medical water-based atomizing agent used in a medical field, which is not specifically limited herein.

In the embodiment of the present disclosure, if water is used as a part of the E-liquid, when a user inhales the E-liquid containing water, the water has the characteristics of removing a foreign flavor, reducing residual E-liquid substances in mouth of the user, providing a purer taste, lowering an atomization temperature and reducing a viscosity. Moreover, since the water has a low viscosity, the viscosity of the E-liquid added with the water is also reduced, so that the user may get more refreshing and less sticky taste experience when inhaling. In addition, the E-liquid containing water can produce smoke by low-temperature heating, which can further reduce the production of harmful substances, and is beneficial for the health of the user, thus realizing a wider application range of this E-liquid in a future electronic atomization field.

The water-based atomizing agent of the embodiment of the present disclosure, and test results and technical effects thereof are described in detail hereinafter through experiments on the water-based atomizing agent containing different mass ratios of ingredients.

It should be noted that, measurement results of harmful substances of various ingredients in the water-based atomizing agent, and experimental results of surface tension and smoke volume of various embodiments will be described in detail in the after-mentioned Table 1 to Table 3.

In addition, in all of the following Embodiment 1 to Embodiment 23, the water matrix of the water-based atomizing agent is a mixture of water and xylitol, the alcohol matrix is selected from glycerol and the propylene glycol, and the active agent is ethanol.

### Embodiment 1

In S1, xylitol was dissolved in water, wherein the mass ratio of xylitol to water was 40:60.

In S2, propylene glycol was evenly mixed with the water matrix (which was namely a mixture of xylitol and water) in the step S1, wherein the mass ratio of propylene glycol to the water matrix in the step S1 was 90:10.

In S3, ethanol was evenly mixed with the mixed matrix (which was namely a mixture of xylitol, water, and propylene glycol) in the step S2 to obtain a water-based atomizing agent, wherein the mass percentage of the ethanol in the obtained water-based atomizing agent was 0.01%.

### Embodiment 2

In S1, xylitol was dissolved in water, wherein the mass ratio of xylitol to water was 40:60.

In S2, glycerol was evenly mixed with the water matrix (which was namely a mixture of xylitol and water) in the step S1, wherein the mass ratio of glycerol to the water matrix in the step S1 was 90:10.

In S3, ethanol was evenly mixed with the mixed matrix (which was namely a mixture of xylitol, water, and glycerol) in the step S2 to obtain a water-based atomizing agent, wherein the mass percentage of the ethanol in the obtained water-based atomizing agent was 5%.

### Embodiment 3

In S1, xylitol was dissolved in water, wherein the mass ratio of xylitol to water was 1:99.

In S2, propylene glycol was evenly mixed with the water matrix (which was namely a mixture of xylitol and water) in the step S1, wherein the mass ratio of propylene glycol to the water matrix in the step S1 was 6:94.

In S3, ethanol was evenly mixed with the mixed matrix (which was namely a mixture of xylitol, water, and propylene glycol) in the step S2 to obtain a water-based atomizing agent, wherein the mass percentage of the ethanol in the obtained water-based atomizing agent was 20%.

### Embodiment 4

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix (which was namely a mixture of xylitol and water, wherein the water matrix in the following embodiments were all the mixture of xylitol and water) in the step S1 was evenly mixed with the alcohol matrix (which was namely a mixture of propylene glycol and glycerol) in the step 2, wherein the mass ratio of the alcohol matrix in the step S2 to the water matrix in the step S1 was 10:90.

In S4, ethanol was evenly mixed with the mixed matrix in the step S3 to obtain a water-based atomizing agent, wherein the mass percentage of the ethanol in the obtained water-based atomizing agent was 0.05%.

### Embodiment 5

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix (which was namely a mixture of propylene glycol and glycerol) in the step 2, wherein the mass ratio of the water matrix in the step S1 to the alcohol matrix in the step S2 was 90:10.

In S4, ethanol was evenly mixed with the mixed matrix in the step S3 to obtain a water-based atomizing agent, wherein the mass percentage of the ethanol in the obtained water-based atomizing agent was 15%.

### Embodiment 6

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 90:10.

In S4, ethanol was evenly mixed with the mixed matrix in the step S3 to obtain a water-based atomizing agent, wherein the mass percentage of the ethanol in the obtained water-based atomizing agent was 0.1%.

### Embodiment 7

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 90:10.

In S4, ethanol was evenly mixed with the mixed matrix in the step S3 to obtain a water-based atomizing agent, wherein the mass percentage of the ethanol in the obtained water-based atomizing agent was 10%.

### Embodiment 8

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 90:10.

In S4, ethanol was evenly mixed with the mixed matrix in the step S3 to obtain a water-based atomizing agent, wherein the mass percentage of the ethanol in the obtained water-based atomizing agent was 0.2%.

### Embodiment 9

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 90:10.

In S4, ethanol was evenly mixed with the mixed matrix in the step S3 to obtain a water-based atomizing agent, wherein the mass percentage of the ethanol in the obtained water-based atomizing agent was 5%.

### Embodiment 10

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 90:10.

In S4, ethanol was evenly mixed with the mixed matrix in the step S3 to obtain a water-based atomizing agent, wherein the mass percentage of the ethanol in the obtained water-based atomizing agent was 0.5%.

### Embodiment 11

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 90:10.

In S4, ethanol was evenly mixed with the mixed matrix in the step S3 to obtain a water-based atomizing agent, wherein the mass percentage of the ethanol in the obtained water-based atomizing agent was 3%.

### Embodiment 12

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 90:10.

In S4, ethanol was evenly mixed with the mixed matrix in the step S3 to obtain a water-based atomizing agent, wherein the mass percentage of the ethanol in the obtained water-based atomizing agent was 2%.

### Embodiment 13

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 90:10.

In S4, ethanol was evenly mixed with the mixed matrix in the step S3 to obtain a water-based atomizing agent, wherein the mass percentage of the ethanol in the obtained water-based atomizing agent was 1%.

### Embodiment 14

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 20:80.

In S4, ethanol was evenly mixed with the mixed matrix in the step S3 to obtain a water-based atomizing agent, wherein the mass percentage of the ethanol in the obtained water-based atomizing agent was 0.5%.

### Embodiment 15

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 30:70.

In S4, ethanol was evenly mixed with the mixed matrix in the step S3 to obtain a water-based atomizing agent, wherein the mass percentage of the ethanol in the obtained water-based atomizing agent was 2%.

### Embodiment 16

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 40:60.

In S4, ethanol was evenly mixed with the mixed matrix in the step S3 to obtain a water-based atomizing agent, wherein the mass percentage of the ethanol in the obtained water-based atomizing agent was 3%.

### Embodiment 17

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 50:50.

In S4, ethanol was evenly mixed with the mixed matrix in the step S3 to obtain a water-based atomizing agent, wherein the mass percentage of the ethanol in the obtained water-based atomizing agent was 4%.

### Embodiment 18

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 60:40.

In S4, ethanol was evenly mixed with the mixed matrix in the step S3 to obtain a water-based atomizing agent, wherein the mass percentage of the ethanol in the obtained water-based atomizing agent was 5%.

### Embodiment 19

In S1, xylitol was dissolved in water, wherein the mass ratio of xylitol to water was 40:60.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 70:30.

In S4, ethanol was evenly mixed with the mixed matrix in the step S3 to obtain a water-based atomizing agent, wherein the mass percentage of the ethanol in the obtained water-based atomizing agent was 6%.

### Embodiment 20

In S1, xylitol was dissolved in water, wherein the mass ratio of xylitol to water was 10:90.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 50:50.

In S4, ethanol was evenly mixed with the mixed matrix in the step S3 to obtain a water-based atomizing agent, wherein the mass percentage of the ethanol in the obtained water-based atomizing agent was 4%.

### Embodiment 21

In S1, xylitol was dissolved in water, wherein the mass ratio of xylitol to water was 20:80.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 50:50.

In S4, ethanol was evenly mixed with the mixed matrix in the step S3 to obtain a water-based atomizing agent, wherein the mass percentage of the ethanol in the obtained water-based atomizing agent was 2%.

### Embodiment 22

In S1, xylitol was dissolved in water, wherein the mass ratio of xylitol to water was 30:70.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 50:50.

In S4, ethanol was evenly mixed with the mixed matrix in the step S3 to obtain a water-based atomizing agent, wherein the mass percentage of the ethanol in the obtained water-based atomizing agent was 3%.

### Embodiment 23

In S1, xylitol was dissolved in water, wherein the mass ratio of xylitol to water was 33:67.

In S2, propylene glycol and glycerol were evenly mixed, wherein the mass ratio of propylene glycol to glycerol was 50:50.

In S3, the water matrix in the step S1 was evenly mixed with the alcohol matrix in the step 2, wherein the mass ratio of the water matrix in the step S1 to the alcohol matrix in the step S2 was 50:50.

In S4, ethanol was evenly mixed with the mixed matrix in the step S3 to obtain a water-based atomizing agent, wherein the mass percentage of the ethanol in the obtained water-based atomizing agent was 3%.

In Embodiment 1 to Embodiment 23 above, since propylene glycol has solvent properties, such as the ability to dissolve a spice in E-liquid, when propylene glycol is added in the E-liquid containing xylitol and water, it can provide a throat hit sensation for the user and has sterilization effect. Therefore, propylene glycol is added in the E-liquid containing water and xylitol.

In Embodiment 1 to Embodiment 23 above, assuming that the water-based atomizing agent is heated by a heating assembly, after water and xylitol are mixed, since water has a relatively large surface tension, the water-based atomizing agent containing water and xylitol may be subjected to resistance of the surface tension of the water, so that it is difficult to heat and atomize the water-based atomizing agent containing water and xylitol by the heating assembly.

However, the surface tension of the water is reduced due to the addition of other substances, so as to reduce the resistance to the water-based atomizing agent flowing to the atomizing core, so that the water-based atomizing agent can flow more quickly and smoothly to the heating assembly, then the heating assembly can more quickly heat and atomize a large amount of water-based atomizing agent containing water and xylitol, thus improving the flowing speed of the water-based atomizing agent flowing to the heating assembly and increasing the production of smoke volume.

It was found that when ethanol was added in the water-based atomizing agent containing water and xylitol, the surface tension of the water could be greatly reduced, so that the heating assembly could fully heat and atomize the E-liquid to produce sufficient smoke volume for the user to inhale.

Further, in the embodiments of the present disclosure, in order to fully dissolve various substances such as the spice in the water-based atomizing agent while reducing the surface tension of the water in the water-based atomizing agent, propylene glycol and ethanol are added in the mixture of xylitol and water.

Further, in the embodiments of the present disclosure, in order to make the water-based atomizing agent produce more smoke during atomization, the alcohol matrix such as glycerol may be added in the water-based atomizing agent.

Tests of the harmful substances, surface tension, and smoke volume of the water-based atomizing agent in Embodiment 1 to Embodiment 23 will be further described.

### I. Measurement of Harmful Substances in Water-Based Atomizing Agent

### Quantitative Analysis Experiment of Carbonyl Compound

According to the newly published national standard GB/T 9733-2008 for atomizing devices, amounts of formaldehyde, acetaldehyde, acrolein, and 2,3-butanedione released in each inhalation of E-liquid were investigated to evaluate the safety of the E-liquid.

Samples selected for experiment were: propylene glycol, glycerol, a blank atomizing agent comprising propylene glycol and glycerol with a mass ratio of 79:21, a control sample 1 comprising propylene glycol and glycerol with a mass ratio of 5:5, a control sample 2 comprising propylene glycol and glycerol with a mass ratio of 4:6, and samples obtained in Embodiment 1 to Embodiment 23.

### Testing Method

2.1) Testing steps of linear smoking machine: the samples as listed above were respectively dripped into an atomizing device provided with an open Rebuildable Dripping Atomizer (RDA), and a heating body, and a E-liquid guide body adapted to different water-based atomizing agents were adjusted according to different surface tensions of various water-based atomizing agents in Embodiment 1 to Embodiment 23 above. Inhalation was carried out by the linear smoking machine (model: SM450) according to a bell-shaped curve, and when puffing, an external constant voltage power supply of the atomizing device was 4.5 V. A puff profile of the inhalation mechanism was a square wave curve, wherein a puff volume was 55 mL, a puff duration was 3 seconds per inhalation with an interval of 30 seconds, and there were 30 inhalations. The smoke volume was captured with two 45 mm Cambridge glass fiber filters (containing an aldehyde ketone derivatization reagent).

2.2) High-performance liquid chromatography test: the contents of major carbonyl compounds in a captured substance were tested by using an industrial standard method of “Cigarettes Determination of major carbonyls in mainstream cigarette smoke”.

2.3) Synchronization steps of atomizing device with smoking machine: a traditional atomizer (resistance wire+E-liquid guide cotton) was synchronized with the smoking machine by using a regulated power supply module (with a maximum voltage of 6.2 V and a test voltage of 4 V).

Test results were as follows:

TABLE 1
Aldehyde and ketone content in smoke generated by traditional
atomizer with E-liquids of propylene glycol, glycerol,
blank atomizing agent, samples obtained in Embodiment 1
to Embodiment 23, and control samples (ng/inhalation)

| | Formalde- | Acetalde- | | 2,3- |
|---|---|---|---|---|
| Sample number | hyde | hyde | Acrolein | butanedione |
| Propylene glycol | 139.60 | 34.8 | 38.67 | 13.5 |
| Glycerol | 234.3 | 78.45 | 69.34 | 23.53 |
| Blank atomizing agent | 123.32 | 46.54 | 43.00 | 15.72 |
| Embodiment 1 | 102.20 | 30.00 | 31.53 | 9.62 |
| Embodiment 2 | 189.21 | 48.09 | 57.52 | 17.87 |
| Embodiment 3 | N.D. | N.D. | N.D. | N.D. |
| Embodiment 4 | N.D. | N.D. | N.D. | N.D. |
| Embodiment 5 | N.D. | N.D. | N.D. | N.D. |
| Embodiment 6 | N.D. | N.D. | N.D. | N.D. |
| Embodiment 7 | N.D. | N.D. | N.D. | N.D. |
| Embodiment 8 | N.D. | N.D. | N.D. | N.D. |
| Embodiment 9 | N.D. | N.D. | N.D. | N.D. |
| Embodiment 10 | N.D. | N.D. | N.D. | N.D. |
| Embodiment 11 | N.D. | N.D. | N.D. | N.D. |
| Embodiment 12 | N.D. | N.D. | N.D. | N.D. |
| Embodiment 13 | N.D. | N.D. | N.D. | N.D. |
| Embodiment 14 | 73.73 | 9.98 | N.D. | N.D. |
| Embodiment 15 | 73.73 | 9.98 | N.D. | N.D. |
| Embodiment 16 | N.D. | N.D. | N.D. | N.D. |
| Embodiment 17 | N.D. | N.D. | N.D. | N.D. |
| Embodiment 18 | N.D. | N.D. | N.D. | N.D. |
| Embodiment 19 | N.D. | N.D. | N.D. | N.D. |
| Embodiment 20 | N.D. | N.D. | N.D. | N.D. |
| Embodiment 21 | N.D. | N.D. | N.D. | N.D. |
| Embodiment 22 | N.D. | N.D. | N.D. | N.D. |
| Embodiment 23 | N.D. | N.D. | N.D. | N.D. |
| Control sample 1 | 102.67 | 22.3 | 41.26 | 32.11 |
| Control sample 2 | 139.00 | 56.14 | 45.67 | 38.72 |

Note:
N.D. = No detected

Data of embodiments in which the mass ratio of glycerol to propylene glycol was 50:50 and the mass ratio of xylitol to water was 40:60 are shown in Table 2.

TABLE 2
Aldehyde and ketone content in smoke generated by traditional
atomizer with E-liquids of different ratios of the
mixture of xylitol and water to the mixture of glycerol
and propylene glycol (ng/inhalation)

| Mixture of xylitol | | | | | |
|---|---|---|---|---|---|
| and water:mixture | | | | | |
| of glycerol and | Proportion | Formal- | Acetal- | Acro- | 2,3- |
| propylene glycol | of alcohol | dehyde | dehyde | lein | butanedione |
| 20:80 | 0.5 | 73.73 | 9.98 | N.D. | N.D. |
| 30:70 | 2 | 73.73 | 9.98 | N.D. | N.D. |
| 40:60 | 3 | N.D. | N.D. | N.D. | N.D. |
| 50:50 | 4 | N.D. | N.D. | N.D. | N.D. |
| 60:40 | 5 | N.D. | N.D. | N.D. | N.D. |
| 70:30 | 6 | N.D. | N.D. | N.D. | N.D. |
| 90:10 | 10 | N.D. | N.D. | N.D. | N.D. |

The detection method of the carbonyl compounds in Table 1 was international GB/T 9733-2008.

It can be seen from the data in Table 1 and Table 2 that, in Embodiment 1 to Embodiment 23 in which there were certain weight ratios of water and xylitol in a total solution, contents of main harmful substances such as formaldehyde, acetaldehyde, acrolein, and 2,3-butanedione, are all far lower than those of formaldehyde, acetaldehyde, acrolein, and 2,3-butanedione in the control sample 1 and the control sample 2. Moreover, Table 2 more intuitively shows that, with the increase of water content to a certain proportion, release amounts of the hydroxyl compounds have been reduced below testing thresholds of the testing equipment. This is because the water-based atomizing agent in the embodiments of the present disclosure contains water and xylitol, and water has a low boiling point, the atomization temperature of the water-based atomizing agent can be greatly reduced. However, the harmful substances are usually produced after the water-based atomizing agent is heated at a high temperature, and hardly produced under low-temperature heating, or ultrasonic vibration of the ultrasonic atomizing assembly under normal-temperature or low-temperature. Even if the harmful substances are produced, the contents of the harmful substances produced may be very low under the low-temperature heating, or ultrasonic vibration of the ultrasonic atomizing assembly under normal-temperature or low-temperature.

Therefore, based on the aforementioned embodiments and the experimental analysis of harmful substances, it is evident that the E-liquid containing water, especially the E-liquid rich in water, is much safer for human use compared with E-liquid used in conventional atomizing devices currently available on the market. The E-liquid containing water should be researched and developed to be more widely used in the field of electronic atomization.

### II. Measurement of Surface Tension

### Comparison of Physical and Chemical Properties of Atomizing Agent of Atomizing Device

The samples of Embodiment 1 to Embodiment 23 were selected for comparison with the blank atomizing agent, and surface tensions of the atomizing agents at normal temperature and normal pressure were determined, respectively.

TABLE 3
Comparison of physical and chemical properties between
water-based atomizing agent and blank atomizing agent

| Number | Surface tension (×1000 N · m⁻¹) |
|---|---|
| Embodiment 1 | 37.83 |
| Embodiment 2 | 48.14 |
| Embodiment 3 | 37.68 |
| Embodiment 4 | 47.76 |
| Embodiment 5 | 32.91 |
| Embodiment 6 | 67.48 |
| Embodiment 7 | 54.23 |
| Embodiment 8 | 67.03 |
| Embodiment 9 | 53.23 |
| Embodiment 10 | 65.48 |
| Embodiment 11 | 56.72 |
| Embodiment 12 | 59.43 |
| Embodiment 13 | 62.38 |
| Embodiment 14 | 39.21 |
| Embodiment 15 | 38.34 |
| Embodiment 16 | 36.82 |
| Embodiment 17 | 37.08 |
| Embodiment 18 | 35.29 |
| Embodiment 19 | 35.73 |
| Embodiment 20 | 38.93 |
| Embodiment 21 | 38.40 |
| Embodiment 22 | 35.69 |
| Embodiment 23 | 35.30 |
| Blank atomizing agent | 35.40 |

The detection method of the surface tensions in Table 3 was national standard: GB/T 5549-2010.

According to the data in Table 3, the water-based atomizing agent with a high water content has higher surface tension compared to blank atomizing agents. This can lead to slower flow rates of the water-based atomizing agent flowing to the atomizing core, thus increasing the airflow resistance in the airway of the atomizing devices. However, by adding the active agent such as ethanol that can reduce surface tension, the surface tension of the water-based atomizing agent rich in water may be adjusted to an appropriate range, so that the water-based atomizing agent can flow more smoothly and E-liquid shortage around the heating body can be greatly reduced, thus enabling the production of more smoke and facilitating the industrial design and production of suitable atomizing devices.

According to the data in Table 3, data of embodiments in which the mass ratio of glycerol to propylene glycol was 50:50, and the mass ratio of xylitol to water was 10:90 were compiled as shown in Table 4.

TABLE 4
Comparison of physical and chemical properties of water-based atomizing
agent comprising glycerol and propylene glycol with a mass ratio
of 50:50, and xylitol and water with a mass ratio of 10:90

| | Mixture of xylitol and | | |
|---|---|---|---|
| | water:mixture of glycerol | Proportion of | Surface |
| Number | and propylene glycol | alcohol % | tension |
| Embodiment 6 | 90:10 | 0.1 | 67.48 |
| Embodiment 8 | 90:10 | 0.2 | 67.03 |
| Embodiment 10 | 90:10 | 0.5 | 65.48 |
| Embodiment 13 | 90:10 | 1 | 62.38 |
| Embodiment 12 | 90:10 | 2 | 59.43 |
| Embodiment 9 | 90:10 | 5 | 53.23 |
| Embodiment 7 | 90:10 | 10 | 54.23 |
| Embodiment 5 | 90:10 | 15 | 32.91 |

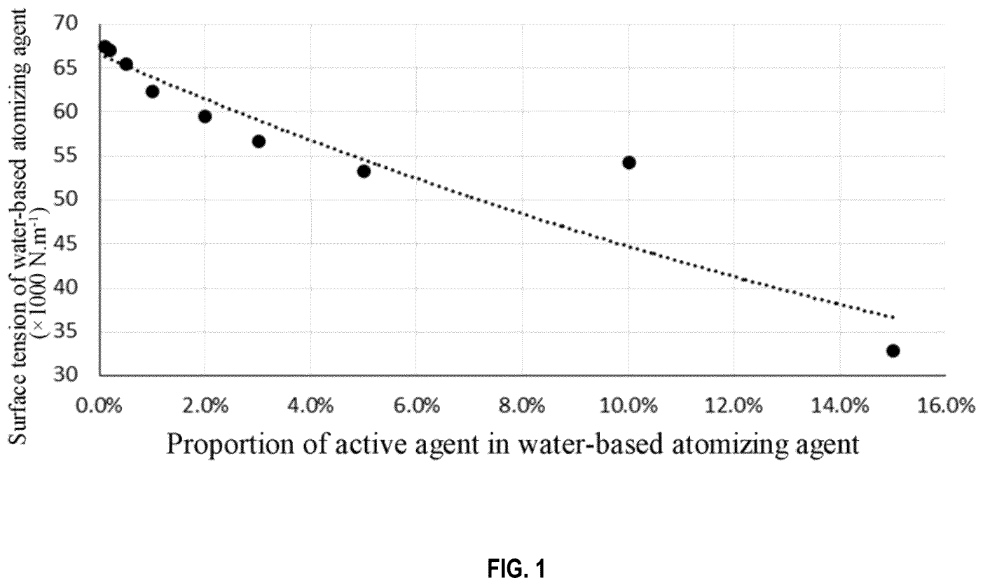

For convenience of understanding, please refer to FIG. 1, which is a diagram showing a relationship between the surface tension of the water-based atomizing agent and the proportion of the active agent in the water-based atomizing agent according to the embodiments of the present disclosure. As shown in FIG. 1, with the increase of the proportion of the active agent such as ethanol in the water-based atomizing agent, the overall surface tension of the water-based atomizing agent may become smaller. In other words, when the active agent such as ethanol accounts for a small proportion in the water-based atomizing agent, the overall surface tension of the water-based atomizing agent is very large, and the surface tension of the water-based atomizing agent can be reduced continuously after adding a certain amount of active agent. There is an inversely proportional relationship between the proportion of the active agent and the surface tension of the water-based atomizing agent. As can be known from Table 3, Table 4, and FIG. 1, in the embodiments of the present disclosure, in the water-based atomizing agent, the active agent plays a key role in producing sufficient smoke with a refreshing taste and without burnt smell.

### III. Measurement of Smoke Volume

### Comparison Test of Smoke Volume

### 1. Experimental Purpose

In order to verify that the xylitol produces a more noticeable amount of smoke in samples with high water content, representative Embodiment 1 to Embodiment 23 and the blank atomizing agent were selected for measurement. Test results of Embodiment 1 to Embodiment 23 and the blank atomizing agent were respectively analyzed independently, considering an interaction of different ingredients in the E-liquid.

### 2. Testing Method

2.1 Testing steps of linear smoking machine: the samples of Embodiment 1 to Embodiment 23 were respectively dripped into an atomizing device provided with an open Rebuildable Dripping Atomizer (RDA), the heating body and a E-liquid guide body in different atomizing cores were adjusted for adaption according to different surface tensions of the E-liquid in various embodiments, and inhalation was carried out by the linear smoking machine (model: SM450) according to a bell-shaped curve. During inhalation, the atomizing device was connected to an external constant voltage power supply at 4.5 V A puff profile of the inhalation mechanism was a square wave curve, wherein an inhalation capacity was 55 mL, a puff duration was 3 seconds per inhalation with an interval of 30 seconds, and there were 30 inhalations, and an average value was calculated. The mass of the atomizing device at an initial stage was weighed by a precision electronic balance, and the mass of smoke volume was characterized according to the weight loss of the atomizing device during inhalation.

### 2.2 Test Results

The test results are shown in the following Table 5.

TABLE 5

| | Total particulate matters |
|---|---|
| Sample number | (mg/inhalation) |
| Embodiment 1 | 0.85 |
| Embodiment 2 | 6.78 |
| Embodiment 3 | 0.12 |
| Embodiment 4 | 2.38 |
| Embodiment 5 | 2.95 |
| Embodiment 6 | 1.75 |
| Embodiment 7 | 2.89 |
| Embodiment 8 | 1.83 |
| Embodiment 9 | 2.66 |
| Embodiment 10 | 2.05 |
| Embodiment 11 | 2.51 |
| Embodiment 12 | 2.34 |
| Embodiment 13 | 2.17 |
| Embodiment 14 | 3.15 |
| Embodiment 15 | 3.42 |
| Embodiment 16 | 4.43 |
| Embodiment 17 | 5.18 |
| Embodiment 18 | 5.72 |
| Embodiment 19 | 5.62 |
| Embodiment 20 | 4.74 |
| Embodiment 21 | 4.98 |
| Embodiment 22 | 5.23 |
| Embodiment 23 | 5.43 |
| Blank atomizing agent | 3.23 |

The total particulate matters in Table 5 above are the smoke volume in the embodiments of the present disclosure, and the total particulate matters are a proper term used to express the smoke volume in experiments in the field of atomization. According to the data in Table 5, in Embodiment 1, the smoke volume is increased obviously with the increase of the proportion of xylitol. Embodiments 15-23 are all superior to the blank atomizing agent in comparison.

As can be clearly seen from Table 5, the addition of xylitol into the water-based atomizing agent rich in water can greatly increase the smoke volume, playing a very significant role in smoke generation.

According to the data in Table 5, the results of the scheme that the mass ratio of glycerol to propylene glycol was 50:50 and the mass ratio of xylitol to water was 50:50 were arranged and shown in Table 6.

TABLE 6

| | Mixture of xylitol and | |
|---|---|---|
| | water:mixture of glycerol | Total particulate |
| | and propylene glycol | matters (mg/inhalation) |
| Embodiment 20 | 10:90 | 4.74 |
| Embodiment 21 | 20:80 | 4.98 |
| Embodiment 22 | 30:70 | 5.23 |
| Embodiment 23 | 33:67 | 5.43 |
| Embodiment 17 | 40:60 | 5.18 |

According to the data in Table 6, the results of the embodiments, in which the mass ratio of glycerol to propylene glycol was 50:50, the mass ratio of xylitol to water was 40:60, and ratios of the mixture of xylitol and water to the mixture of glycerol and propylene glycol were different, were compiled as shown in Table 7.

TABLE 7

| | Mixture of xylitol and | Total number of |
|---|---|---|
| | water:mixture of glycerol | particles |
| | and propylene glycol | (mg/inhalation) |
| Embodiment 14 | 20:80 | 3.15 |
| Embodiment 15 | 30:70 | 3.42 |
| Embodiment 16 | 40:60 | 4.43 |
| Embodiment 17 | 50:50 | 5.18 |
| Embodiment 18 | 60:40 | 5.72 |
| Embodiment 19 | 70:30 | 5.62 |

It can be intuitively seen from Table 6 that, under the same conditions, a total number of particles shows an increasing trend when the proportion of the sugar alcohol in the water matrix is larger. It can be clearly found from Table 7 that, the total number of particles also shows the increasing trend when the proportion of the mixture of xylitol and water in the water matrix is increased. Compared with Embodiment 3 with a high water content, the proportion of the xylitol is small, and data of the total number of particles is at a very low level, thus it can be seen that the water matrix comprising the sugar alcohol such as xylitol plays a decisive role in atomization effect of a water-based atomizing substance.

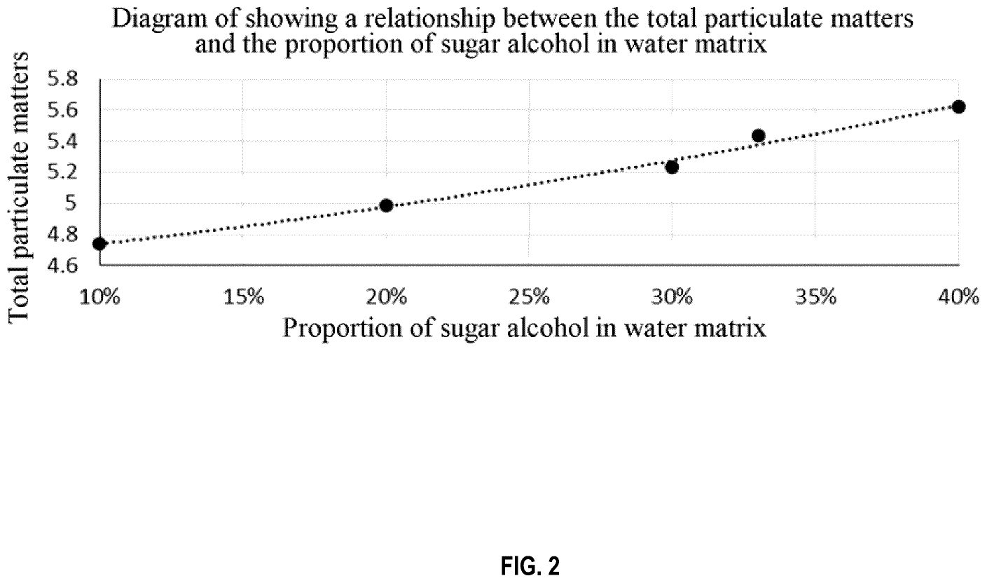

For convenience of understanding, please refer to FIG. 2. FIG. 2 is a diagram showing a relationship between the total particulate matters and the proportion of the sugar alcohol in the water matrix according to the embodiments of the present disclosure. It should be noted that the sugar alcohol in Embodiments 1-23 above is xylitol. It can be seen from FIG. 2 that, with the increase of the proportion of sugar alcohol such as xylitol in the water matrix, the total particulate matters (which are namely the smoke volume) can be increased continuously. In other words, when the sugar alcohol such as xylitol accounts for a very small proportion in the water matrix, there may be a few of total particulate matters (which are namely the smoke volume) produced correspondingly. However, when the proportion of sugar alcohol such as xylitol in the water matrix is increased to a certain extent, the total particulate matters (which are namely the smoke volume) produced can also be increased correspondingly. There is a direct proportional relationship between the proportion of the sugar alcohol and the total particulate matters.

The water-based atomizing agent in the embodiments of the present disclosure has been described in detail above, and the water-based atomizing substance for specific purpose in an embodiment of the present disclosure will be described below.

In the embodiment of the present disclosure, the water-based atomizing substance for specific purpose comprises:
the water-based atomizing agent in any one of the above-mentioned embodiments; and
an additive, wherein the additive is used for personal consumption or a medical purpose.

Further, the additive contains a natural specific ingredient, or the additive contains a synthetic specific ingredient, or the additive contains a natural specific ingredient and a synthetic mixed specific ingredient, which is not specifically limited herein.

Further, the specific ingredient may be an herbal extract.

Further, the specific ingredient is nicotine.

An embodiment of the present disclosure further provides a cartomizer. The cartomizer comprises at least one cartomizer chamber, wherein the cartomizer chamber is filled with the water-based atomizing agent in any one of the above-mentioned embodiments, or the water-based atomizing substance for specific purpose in any one of the above-mentioned embodiments.

In addition, an embodiment of the present disclosure further provides an atomizing device. The atomizing device comprises the cartomizer in the above-mentioned embodiment. Preferably, the atomizing device is a medical instrument or a personal consumption appliance; and more preferably, the personal consumption appliance is an electronic cigarette. It should be noted that, the medical instrument may be a medical instrument used for treating oropharynx, trachea, and lung diseases, or a medical instrument used for cosmetology, which is not specifically limited herein.

As described above, the embodiments above are only used to illustrate the technical solutions of the present disclosure, and are not intended to limit the present disclosure. Although the present disclosure has been described in detail with reference to the above-mentioned embodiments, those of ordinary skills in the art should understand that the technical solutions set forth by the above-mentioned embodiments can still be modified, or equivalent substitutions can be made to part of the technical features in the embodiments. However, these modifications or substitutions should not depart from the spirit and scope of the technical solutions of the embodiments of the present disclosure.

What is claimed is:

1. A water-based atomizing agent, comprising:
a mixed matrix; and
an active agent, wherein the active agent is used for improving a surface activity of the mixed matrix.

2. The water-based atomizing agent according to claim 1, wherein the mixed matrix comprises a water matrix and an alcohol matrix.

3. The water-based atomizing agent according to claim 2, wherein the alcohol matrix is polyol, and the polyol comprises ethylene glycol, propylene glycol, glycerol, pentaerythritol, or a combination thereof.

4. The water-based atomizing agent according to claim 3, wherein the alcohol matrix is propylene glycol, glycerol, or a combination thereof.

5. The water-based atomizing agent according to claim 2, wherein the water matrix comprises water and sugar alcohol, and the sugar alcohol is erythritol, xylitol, mannitol, sorbitol, or a combination thereof.

6. The water-based atomizing agent according to claim 2, wherein the water matrix comprises water and sugar alcohol, and the sugar alcohol is xylitol.

7. The water-based atomizing agent according to claim 1, wherein:
a mass percentage of the active agent in the water-based atomizing agent is 0.01% to 20%.

8. The water-based atomizing agent according to claim 5, wherein a mass percentage of the active agent in the water-based atomizing agent is 0.2%-5%.

9. The water-based atomizing agent according to claim 6, wherein a mass percentage of the active agent in the water-based atomizing agent is 0.2%-5%.

10. The water-based atomizing agent according to claim 2, wherein the mixed matrix comprises:
10 to 94 parts by weight of water matrix; and
6 to 90 parts by weight of alcohol matrix.

11. The water-based atomizing agent according to claim 2, wherein the mixed matrix comprises:
30 to 80 parts by weight of water matrix; and
20 to 70 parts by weight of alcohol matrix.

12. The water-based atomizing agent according to claim 5, wherein the water matrix comprises:
60% to 99% by mass percentage of water and 1% to 40% by mass percentage of sugar alcohol.

13. The water-based atomizing agent according to claim 6, wherein the water matrix comprises:
60% to 70% by mass percentage of water and 30% to 40% by mass percentage of sugar alcohol.

14. The water-based atomizing agent according to claim 1, wherein a surface tension of the active agent is smaller than that of the mixed matrix; and
the active agent is at least one selected from the group consisting of phenethyl alcohol, ethyl lactate, ethyl acetate, and ethanol.

15. The water-based atomizing agent according to claim 1, wherein a surface tension of the active agent is smaller than that of the mixed matrix; and the active agent is ethanol.

16. The water-based atomizing agent according to claim 1, wherein the water-based atomizing agent is in a liquid state, a semi-solid state, or a solid state.

17. A water-based atomizing substance, comprising:
a water-based atomizing agent, comprising a mixed matrix and an active agent, wherein the active agent is used for improving a surface activity of the mixed matrix; and
an additive, wherein the additive is used for personal consumption or a medical purpose.

18. The water-based atomizing substance according to claim 17, wherein the additive comprises a natural or synthetic specific ingredient,
wherein the synthetic specific ingredient is an herbal extract; or
the synthetic specific ingredient is nicotine or a derivative thereof.

19. The water-based atomizing substance according to claim 17, wherein the additive comprises a natural and synthetic specific ingredient,
wherein the synthetic specific ingredient is an herbal extract; or
the synthetic specific ingredient is nicotine or a derivative thereof.

20. A cartomizer, comprising at least one cartomizer chamber, wherein the cartomizer chamber is filled with a water-based atomizing agent, the water-based atomizing agent comprising:
a mixed matrix; and
an active agent, wherein the active agent is used for improving a surface activity of the mixed matrix.
